(19) United States
(12) Patent Application Publication
(10) Pub. No.: US 2010/0030319 A1
(43) Pub. Date: Feb. 4, 2010

(54) COILS FOR VASCULAR IMPLANTS OR OTHER USES

(75) Inventor: Jan Weber, Maastricht (NL)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/509,050

(22) Filed: Jul. 24, 2009

Related U.S. Application Data
(60) Provisional application No. 61/085,237, filed on Jul. 31, 2008.

(51) Int. Cl. A61F 2/88; B21F 45/00

(52) U.S. Cl. 623/1.11; 623/1.22; 140/71 R

(57) ABSTRACT

Medical devices comprising implants for use in blood vessels or other body lumens. The implant comprises an elongate member that, in its native configuration, follows a generally helical path. The elongate member is formed of one or more strands that are wound into a coil of minor windings, wherein the coil of minor windings is itself wound into the generally helical path. The one or more strands are formed of materials that provide the elongate member with the desired flexibility. In some cases, the elongate member may be capable of delivering a therapeutic agent. This can be accomplished by, for example, using capsules, swellable materials, corrodable elements, magnetically-sensitive particles, coatings, and/or core wires. Also provided are a method of treating a superficial femoral artery and a method of making an implant.

### RELATED APPLICATIONS

This application claims the benefit of U.S. Provisional Application Ser. No. 61/085,237 (filed 31 Jul. 2008), which is incorporated by reference herein. This application is also related to and incorporates by reference U.S. Provisional Application Ser. No. 61/228,264 (filed 24 Jul. 2009), entitled “Medical Devices Having an Inorganic Coating Layer Formed by Atomic Layer Deposition” by applicants Jan Weber and Aiden Flanagan.

### TECHNICAL FIELD

The present invention relates to medical devices that can be implanted in blood vessels or other body lumens.

### BACKGROUND

Vascular stents are now widely used in interventional procedures for treating occlusions in the coronary arteries and other blood vessels. Vascular stents generally have a tubular shape and are deployed in a blood vessel to restore and maintain patency of a diseased segment of the blood vessel. More recently, vascular stents have been used in combination with local drug delivery to prevent restenosis in the vessel.

Vascular stents are most commonly used in the coronary arteries, but recent efforts have focused on the use of stents to treat other arteries, such as the superficial femoral artery. However, conventional vascular stents have had mixed success when used in these other blood vessels. Vascular stents for use in these other blood vessels require a different set of structural characteristics than those conventionally used for coronary artery stenting. Therefore, there is a need for devices and methods for treating a wider range of blood vessels, including the superficial femoral arteries, as well as other body lumens.

### SUMMARY

In one aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; and a plurality of capsules disposed in the lumen of the coil of minor windings, wherein the capsules contain a therapeutic agent. In some cases, the coil of minor windings comprises gaps between the minor windings, and the size of the capsules is larger than the size of the gaps between the minor windings. In some cases, the medical device further comprises magnetite particles contained in the capsules. In some cases, the medical device further comprises a magnetic element disposed in the lumen of the coil of minor windings. In some cases, the medical device further comprises a swellable material disposed in the lumen of the coil of minor windings. In some cases, swelling of the swellable material causes the capsules to collapse. In some cases, the medical device further comprises a corrodable element disposed in the lumen of the coil of minor windings, and the swelling of the swellable material is pH-dependent. In some cases, the corrodable element comprises magnesium.

In some cases, the strand comprises a biocompatible metallic material. In some cases, the medical device further comprises a cap covering the lumen at each end of the coil of minor windings. In some cases, the medical device further comprises a delivery catheter having a catheter lumen, wherein the implant is contained in the catheter lumen. In some cases, when the implant is in the catheter lumen, the coil of minor windings is in an extended configuration. In some cases, when the implant is in the catheter lumen, the coil of minor windings is in a folded configuration. In some cases, the width of each fold of the coil of minor windings in the folded configuration is substantially the same as the width of each of the major windings. In some cases, when the implant is in the catheter lumen, the coil of minor windings is in a compact coiled configuration. In some cases, the capsules further contain a swellable material.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; and wherein the lumen of the coil of minor windings is separated into compartments. In some cases, the medical device further comprises a plurality of lumen barriers that separate the lumen of the coil of minor windings into the compartments. In some cases, the strand comprises a biocompatible metallic material. In some cases, the medical device further comprises a cap covering the lumen at each end of the coil of minor windings. In some cases, the medical device further comprises a delivery catheter having a catheter lumen, wherein the implant is contained in the catheter lumen. In some cases, when the implant is in the catheter lumen, the coil of minor windings is in an extended configuration. In some cases, when the implant is in the catheter lumen, the coil of minor windings is in a folded configuration. In some cases, the width of each fold of the coil of minor windings in the folded configuration is substantially the same as the width of each of the major windings. In some cases, when the implant is in the catheter lumen, the coil of minor windings is in a compact coiled configuration.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; and a core wire disposed in the lumen of the coil of minor windings, wherein the core wire biases the coil of minor windings such that the axis of the minor windings follow the generally helical path; wherein the improvement comprises a coating disposed on the core wire, wherein the coating comprises a therapeutic agent.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; and a core wire disposed in the lumen of the coil of minor windings, wherein the core wire biases the coil of minor windings such that the axis of the minor windings follow the generally helical path; wherein the improvement comprises the core wire being comprised of a biodegradable polymer. In some cases, a coating comprising a therapeutic agent is disposed over the core wire.

In another aspect, the present invention provides a method of treating a superficial femoral artery comprising: providing a medical device, wherein the medical device comprises an implant comprising a strand wound into a coil of minor windings about an axis, wherein the minor windings define a lumen that contains a therapeutic agent, and wherein the coil of minor windings is wound into a coil of major windings such that the axis of the minor windings follows a generally helical path; and implanting the implant in the superficial femoral artery.

In another aspect, the present invention provides a method of making an implant comprising: providing (a) a strand wound into a coil of minor windings, wherein the minor windings define a lumen; and (b) a core wire that is biased towards a generally helical configuration, wherein the core wire is disposed in the lumen of the coil of minor windings, and wherein the improvement comprises: holding the core wire in an extended configuration, wherein the length of the core wire in the extended configuration is greater than the length of the coil; coating a first portion of the core wire with a therapeutic agent; disposing the first portion of the core wire inside the lumen of the coil; cutting off a portion of the core wire that is not inside the lumen of the coil; and affixing the core wire to the strand. In some cases, the method further comprises disposing a second portion of the core wire inside the lumen of the coil prior to the step of coating the first portion. In some cases, the step of disposing the first portion of the core wire inside the lumen of the coil comprises either: (a) sliding the coil from the second portion to the first portion of the core wire; (b) sliding the core wire to move the first portion of the core wire into the lumen of the coil; or both.

In another aspect, the present invention provides a medical device comprising: an implant comprising a strand wound into a coil of minor windings about two or more axes such that the minor windings define at least first and second lumens, and wherein the coil of minor windings is wound into a coil of major windings such that each of the axes of the minor windings follows a generally helical path. In some cases, the strand is wound along a figure-8 path. In some cases, a therapeutic agent is contained in at least one of the first or second lumens defined by the minor windings. In some cases, the therapeutic agent contained in the first lumen is different from the therapeutic agent contained in the second lumen.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; wherein the diameter of the minor windings at a portion of the implant is different from the diameter of the minor windings at another portion of the implant. In some cases, the coil of minor windings has a plurality of narrow regions and a plurality of wide regions. In some cases, the narrow regions seal the wide regions into compartments. In some cases, the medical device further comprises lumen barriers within the lumen defined by the minor windings at the narrow regions. In some cases, the medical device further comprises a core wire disposed in the lumen defined by the minor windings. In some cases, a therapeutic agent is contained in the lumen defined by the minor windings.

In another aspect, the present invention provides a medical device including an implant comprising: a first strand wound into a first coil of minor windings about an axis, wherein the minor windings define a first lumen, and wherein the first coil of minor windings is wound into a first coil of major windings such that the axis of the minor windings follows a generally helical path; and a second strand wound into a second coil of minor windings about an axis, wherein the minor windings define a second lumen, and wherein the second coil of minor windings is wound into a second coil of major windings such that the axis of the minor windings follows a generally helical path. In some cases, the first coil of major windings and the second coil of major windings define a common lumen for at least a portion of the implant. In some cases, the first coil of major windings and the second coil of major windings define different lumens for at least a portion of the implant. In some cases, the first coil of major windings and the second coil of major windings define a common lumen for a portion of the implant, and the first coil of major windings and the second coil of major windings define different lumens for another portion of the implant.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; wherein one or more of the major windings has a flexible portion. In some cases, the generally helical path followed by the axis of the minor windings is interrupted at the flexible portion. In some cases, the path taken by the axis of the minor windings at the flexible portion is limited to a cylindrical plane defined by the major windings.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; wherein the implant serves as an inductor in a resonance circuit. In some cases, the resonance circuit is tuned to resonate at a frequency in the range of 30-300 MHz. In some cases, the medical device further comprises a capacitance structure electrically coupled to the implant. In some cases, the capacitance structure and the implant form a resonance LC circuit. In some cases, the capacitance structure has adjustable capacitance. In some cases, the capacitance structure includes a portion of the implant. In some cases, the portion of the implant serves as an electrode of the capacitance structure. In some cases, the resonance circuit includes a plurality of parallel circuits.

In another aspect, the present invention provides a medical device comprising: an implant comprising a strand wound into a coil of minor windings about an axis, wherein the minor windings define a lumen, and wherein the coil of minor windings is wound into a coil of major windings such that the axis of the minor windings follows a generally helical path; wherein the implant is divided into segments that are electrically isolated from each other. In some cases, the segments are separated from each other by an insulating connector comprising a non-conductive material. In some cases, the length of one or more of the segments is less than 13 cm.

In another aspect, the present invention provides a medical device comprising: a delivery catheter having a catheter lumen; and an implant comprising a strand wound into a coil of minor windings about an axis, wherein the minor windings define a lumen, and wherein the coil of minor windings is wound into a coil of major windings such that the axis of the minor windings follows a generally helical path. The implant may be contained in the catheter lumen of the delivery catheter with the coil of minor windings in an extended configuration, in a folded configuration, or in a compact coiled configuration.

In another aspect, the present invention provides a medical device comprising: a delivery catheter having a catheter lumen; and an implant comprising a strand wound into a coil of minor windings about an axis, wherein the minor windings define a lumen, and wherein the coil of minor windings is wound into a coil of major windings such that the axis of the minor windings follows a generally helical path; wherein the implant is contained in the catheter lumen of the delivery catheter with the coil of minor windings in a compact coiled configuration. In some cases, the compact coiled configuration includes turns in a clockwise direction and turns in a counter-clockwise direction. In some cases, the number of clockwise turns is approximately the same as the number of counter-clockwise turns.

In another aspect, an implant of the present invention comprises a plurality of particles disposed within the lumen of the minor windings, with the particles carrying a therapeutic agent. The particles may comprise an inorganic material. The particles may have an average size that is larger than the size of the gaps between the minor windings of the implant. In some cases, the particles have an average size of 10 μm or greater. In some cases, the coil of minor windings comprises gaps between the minor windings, and the average size of the particles is larger than the size of the gaps between the minor windings. In some cases, the particles are porous, and the therapeutic agent is contained in the pores.

In another aspect, an implant of the present invention has one or more anchors attached thereto. The anchors may serve to secure the implant to body tissue. In some cases, the anchors are biodegradable or bioerodable.

In another aspect, an implant of the present invention has an inner coating disposed on the luminal surface of the coil of minor windings and an outer coating disposed on the external surface of the coil of minor windings. The thickness of the inner coating and the thickness of the outer coating may be substantially the same. The coating may be deposited by atomic layer deposition.

In another aspect, a medical device is made by coating an implant using a self-limiting deposition process, such as atomic layer deposition.

### BRIEF DESCRIPTION OF THE DRAWINGS

FIGS. 1A and 1B show a medical device according to an embodiment of the present invention. FIG. 1A shows a side view of an elongate member. FIG. 1B shows a detailed view of a segment of the elongate member.

FIG. 2 shows a detailed view of a segment of an elongate member according to an embodiment.

FIG. 3 shows a detailed view of a segment of an elongate member according to another embodiment.

FIG. 4A shows a perspective view of a segment of a coil of minor windings of a medical device according to another embodiment. In FIG. 4B, the arrows show the “figure-8” path taken by the strand to form the minor windings in FIG. 4A.

FIGS. 5A and 5B show a medical device according to another embodiment. FIG. 5A shows a side view of an elongate member. FIG. 5B shows a detailed, longitudinal cross-section view of a segment of the elongate member.

FIG. 6 shows a segment of an elongate member according to another embodiment.

FIG. 7A shows a side view of a segment of an implant according to another embodiment. FIG. 7B shows an end view of the implant of FIG. 7A.

FIG. 8 shows a side view of an implant according to another embodiment.

FIGS. 9A and 9B show a medical device according to another embodiment. FIG. 9A shows a detailed, longitudinal cross-section view of a segment of an elongate member, prior to implantation. FIG. 9B shows the segment of FIG. 9A after implantation.

FIGS. 10A and 10B show a medical device according to another embodiment. FIG. 10A shows a detailed, longitudinal cross-section view of a segment of an elongate member, prior to implantation. FIG. 10B shows the segment of FIG. 10A after implantation.

FIGS. 11A-11D show a medical device according to another embodiment. FIG. 11A shows a side view of an elongate member. FIG. 11B shows a detailed view of a segment of the elongate member. FIG. 11C shows a core wire. FIG. 11D shows a transverse cross-section view of the elongate member.

FIG. 12A shows a side view of an implant according to another embodiment. FIG. 12B shows a schematic diagram of the circuit formed in FIG. 12A.

FIG. 13 shows a side view of an implant according to another embodiment.

FIG. 14 shows a side view of an implant according to another embodiment.

FIGS. 15A-15C show a method of making the medical device of FIGS. 11A-11D.

FIG. 16 shows the distal end of a portion of a medical device according to another embodiment (with a see-through view of the delivery catheter).

FIG. 17 shows the distal end of a portion of a medical device according to another embodiment (with a see-through view of the delivery catheter).

FIG. 18 shows a side view of an implant according to another embodiment.

FIGS. 19A-E illustrate an example of how a coating can be formed by atomic layer deposition.

FIGS. 20A-C show an implant having a coating deposited by atomic layer deposition. FIG. 20A shows a side view of a portion of the implant. FIG. 20B shows an end view of the implant portion shown in FIG. 20A. FIG. 20C shows a longitudinal cross-section view of the implant portion shown in FIG. 20A.

FIGS. 21A-F illustrate how an aluminum oxide coating may be formed on an implant by atomic layer deposition.

FIG. 22A is a microscopic image of a 5 nm thick titanium oxide coating on a coronary artery stent. FIG. 22B is a microscopic image of a 30 nm thick titanium oxide coating on a coronary artery stent.

### DETAILED DESCRIPTION

Medical devices of the present invention comprise an implant in the form of an elongate member that, in its native configuration, follows a generally helical path. As used herein, the term “native configuration,” when referring to the elongate member, means the shape in which the elongate member exists in the absence of any deforming stresses. But otherwise, the elongate member is sufficiently flexible that it will generally conform to the anatomy of the body part where it is to be implanted (e.g., by extending, compressing, or bending). For example, where the elongate member is implanted in a blood vessel, it may be deformed from its native configuration to follow the anatomy of the blood vessel. As such, the shape and dimensions of the elongate member may be altered from its native configuration when the elongate member is constrained (such as in a delivery catheter or after implantation in a blood vessel). As used herein, the term “major windings” refers to the windings that are formed by the elongate member following the generally helical path.

The elongate member is formed of one or more strands that are wound into a coil about an axis. As used herein, the term “minor windings” refers to the windings that are formed by the strand being wound into a coil. Thus, in forming the major windings, the axis of the coil of minor windings follows a generally helical path. The coil of minor windings defines one or more lumens. A therapeutic agent may be contained in the one or more lumens.

As used herein, a “strand” is any suitable flexible wire-like structure that can be wound into a coil, including wires, strips, filaments, strings, threads, etc. The transverse cross-section of the strand can have any suitable shape, including circular, rectangular, square, or oval. More than one strand may be used to make the coil. For example, two or more strands may be braided, intertwined, interwoven, etc. Also, two or more strands may be used in series to form the coil (e.g., a strand may be interrupted midway through the coil and the coil is continued using another strand).

The one or more strands are formed of materials that provide the elongate member with the desired flexibility. Such materials include polymers and metals. Further, the materials used in the strands include those that are biocompatible or otherwise known to be used in implantable medical devices. In some cases, the strand comprises a biocompatible metallic material, such as nitinol, iridium, platinum, stainless steel (e.g., 316 L) or a mixture thereof.

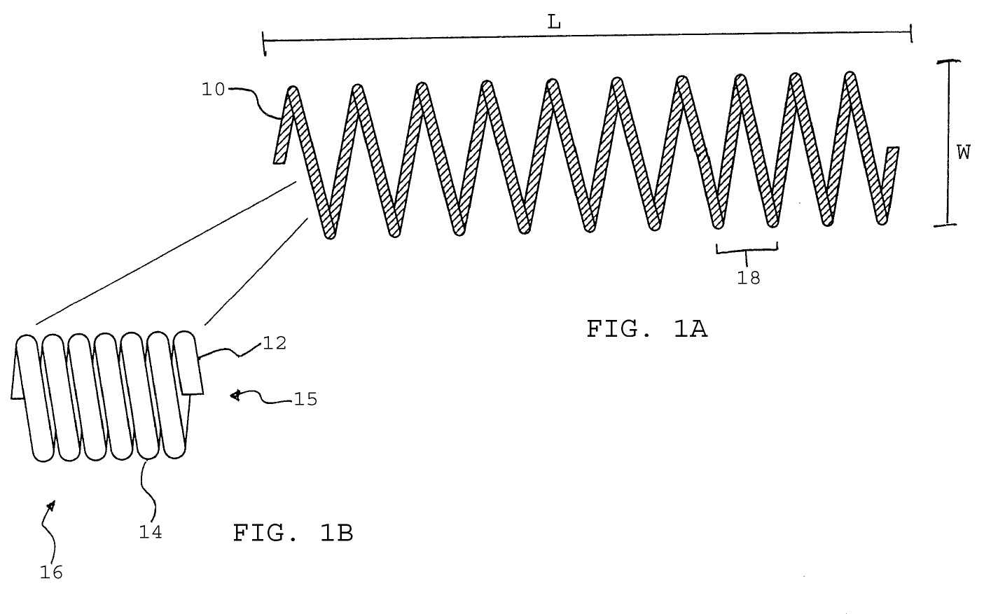

Referring to the embodiment shown in FIGS. 1A and 1B, a medical device comprises an implant in the form of an elongate member 10 that, in its native configuration, follows a generally helical path to form major windings 18. Elongate member 10 is sufficiently flexible that it will conform to the anatomy of the body part where it is to be implanted (e.g., by extending, compressing, or bending). This flexibility can provide elongate member 10 with fatigue resistance and allow it to conform to non-tubular geometries (e.g., vascular bifurcations or aneurysms). As such, the medical device can be useful in the superficial femoral artery, where implanted devices are particularly vulnerable to fatigue failure due to the repeated compression, extension, or bending resulting from hip or leg motion.

The dimensions of the coil formed by elongate member 10 will vary depending upon the particular application. In some cases, the length (L) of the coil formed by elongate member 10 (in its native helical configuration) may be in the range of 2 cm to 40 cm. A coil length in this range is particularly suitable for use in the superficial femoral artery, which can have lesions that extend for many centimeters.

FIG. 1B shows a detailed view of a segment 16 of elongate member 10. As seen in this view, elongate member 10 comprises a wire coil 12 that forms minor windings 14. The dimensions of wire coil 12 will vary depending upon the particular application. In some cases, the diameter (D) of wire coil 12 may be 200 μm or less. Having the diameter of wire coil 12 be in this range can be useful in reducing the amount of intravascular turbulence in cases where the medical device is used in the superficial femoral artery.

Minor windings 14 define a lumen 15 of wire coil 12. In certain embodiments, a therapeutic agent is disposed in lumen 15 of wire coil 12. In some cases, the terminal ends of wire coil 12 are capped to retain the therapeutic agent within lumen 15. The therapeutic agent can be provided in a variety of ways. For example, the therapeutic agent may be mixed with binder or filler materials which serve as a carrier for the therapeutic agent, bind it to the medical device, and/or control the release of the therapeutic agent. The therapeutic agent may be applied by any of various means by which therapeutic agents are applied on medical devices, such as spraying or dipping. The spraying or dipping may be performed with elongate member 10 slightly extended to create gaps within minor windings 14, allowing the fluid to penetrate into lumen 15 of wire coil 12.

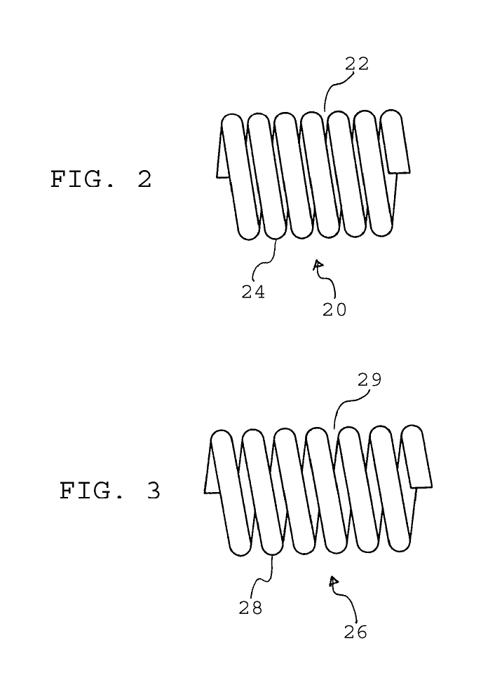

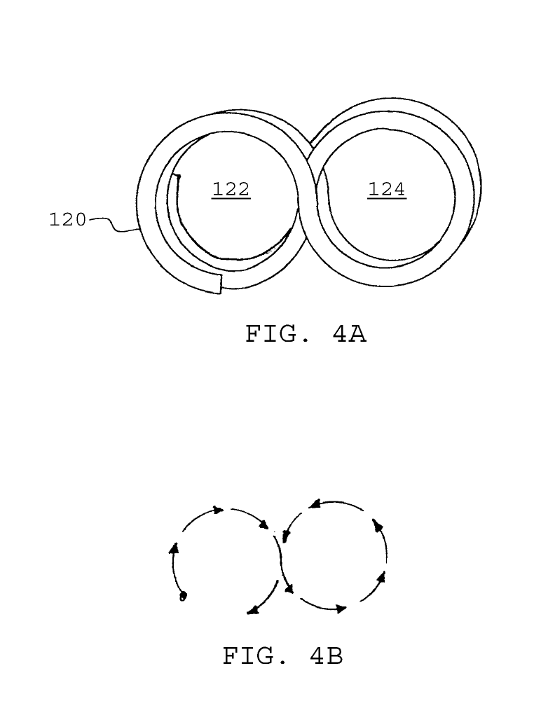

Further, the gaps between windings 14 may be adjusted to control the release of the therapeutic agent. For example, referring to the embodiment shown in FIG. 2, a segment 20 of an elongate member has gaps 22 between minor windings 24 that are relatively narrower to provide a relatively slower release of the therapeutic agent. Referring to the embodiment shown in FIG. 3, a segment 26 of an elongate member has gaps 29 between minor windings 28 that are relatively wider to provide a relatively faster release of the therapeutic agent. The gaps may be adjusted by changing various structural parameters of the wire coil, including changing the thickness of the wire or by changing the pitch of the minor windings.

Also, referring back to FIGS. 1A and 1B, the width of the gaps between minor windings 14 may not necessarily be uniform throughout the length of elongate member 10. Wire coil 12 in some parts of the elongate member may have narrower gaps and other parts may have wider gaps. For example, a middle portion of elongate member 10 may have relatively narrower gaps between minor windings 14 of wire coil 12, whereas the end portions of elongate member 10 may have relatively wider gaps between minor windings 14 of wire coil 12.

Depending upon the path taken by the strand, the coil of minor windings may define a single lumen or multiple lumens (i.e., two or more lumens). In certain embodiments, the minor windings may define multiple lumens. The strand may take various paths (e.g., a “figure-8” path) suitable to form a coil of minor windings having multiple lumens. By having multiple lumens formed by the coil of minor windings in this manner, different therapeutic agents may be provided in the different lumens, or the different lumens may provide different release rates for a therapeutic agent.

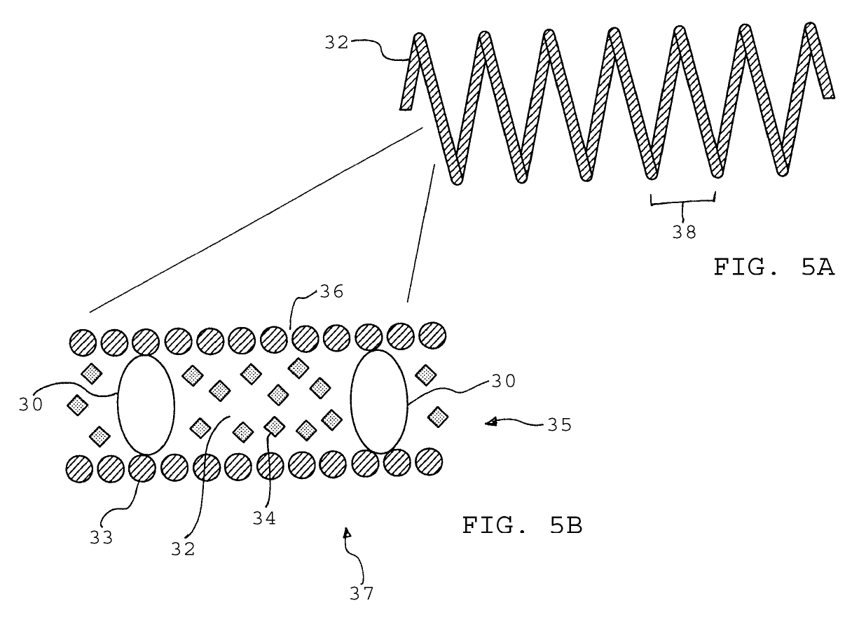

Referring to the embodiment shown in FIGS. 4A and 4B, the strand may take a “figure-8” path to form a coil of minor windings having two lumens. FIG. 4A shows a strand 120 wound in such a manner as to form a coil of minor windings having two lumens, 122 and 124. In FIG. 4B, the arrows show the “figure-8” path taken by strand 120 to form the minor windings.

In some cases, lumens 122 and 124 contain one or more therapeutic agents. For example, each of lumens 122 and 124 may contain a different therapeutic agent, or alternatively, the same therapeutic agent may be contained in both lumens, but released at different rates.

Upon the implantation of elongate member 10 at the body site, it is possible that the gaps between minor windings 14 become wider (relative to the native configuration) when it is subject to deformative stresses. As a result, release of the therapeutic agent through this enlarged gap may be faster than intended. Thus, to control the amount of therapeutic agent released through this enlarged gap, in certain embodiments, lumen 15 of wire coil 12 may be separated into compartments. In such embodiments, only the therapeutic agent contained in the compartment that encompasses the enlarged gap would be affected.

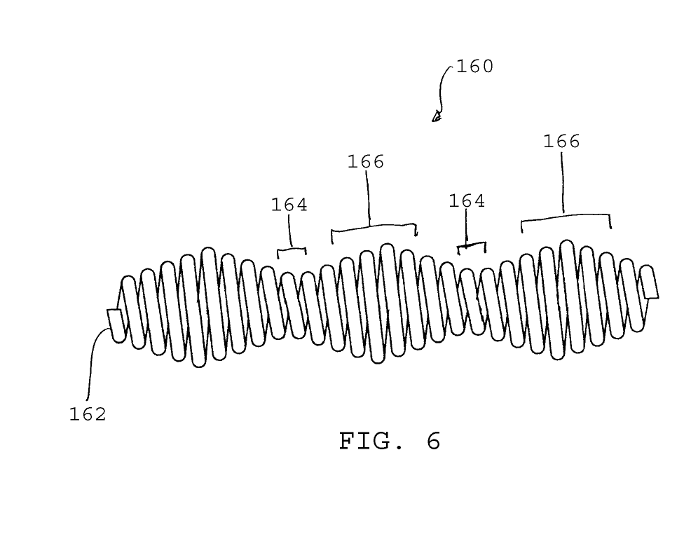

Any of various structures may be used to create compartments within lumen 15 of wire coil 12. For example, a plurality of lumen barriers (e.g., beads) may be positioned inside the lumen at spaced intervals (which may be regular or irregular), with the space between the lumen barriers forming the compartments. Referring to the embodiment shown in FIGS. 5A and 5B, a medical device comprises an implant in the form of an elongate member 32 that, in its native configuration, follows a generally helical path to form major windings 38. FIG. 5B shows a detailed cross-section view of a segment 37 of elongate member 32. As seen in this view, elongate member 32 comprises a wire coil 33 that forms the minor windings. The lumen 35 of wire coil 33, as defined by the minor windings, contains a therapeutic agent 34. Upon implantation, therapeutic agent 34 is released through the gaps 36 between the minor windings.

Further, lumen 35 contains lumen barriers 30 that separate lumen 35 into compartments. In FIG. 5B, the space between lumen barriers 30 defines a compartment 32. As such, if the gaps 36 between the minor windings of wire coil 33 in segment 37 were to become excessively wide, only therapeutic agent 34 within the affected compartment 32 would be released through the widened gap.

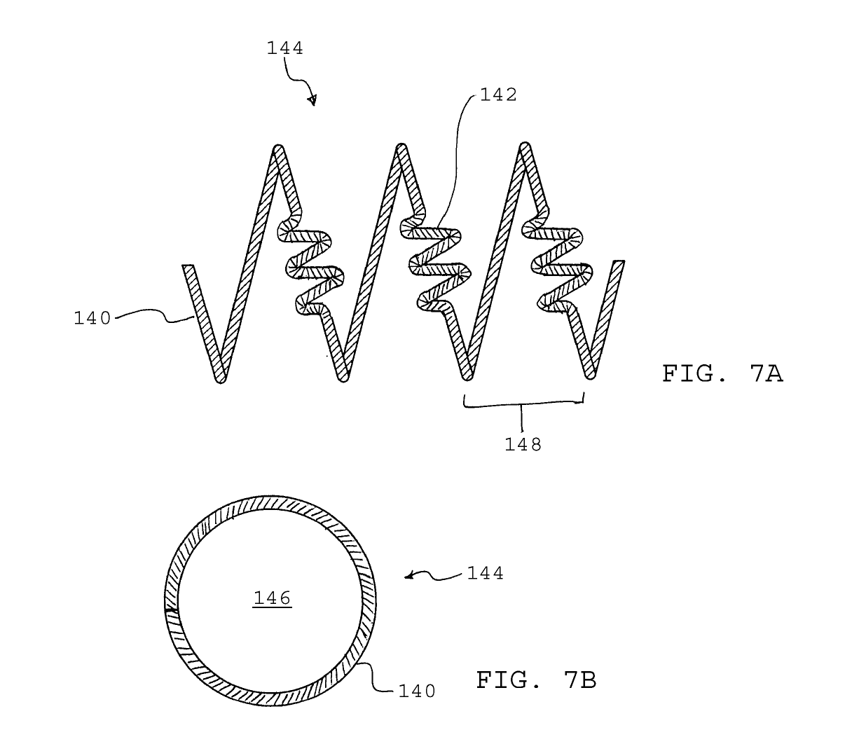

The dimensions of the minor windings are not necessarily uniform throughout the elongate member. For example, the diameter of the minor windings may vary along the length of the elongate member. This feature may be useful in increasing the flexibility of the elongate member. Referring to the embodiment shown in FIG. 6, a segment 160 of an elongate member has a strand 162 that is wound into a coil of minor windings. The diameter of the minor windings varies along the length of the elongate member at segment 160. This variation in the diameter of the minor windings provides narrow regions 164 and wide regions 166 in the coil formed by the minor windings. Narrow regions 164 can provide increased flexibility to the elongate member.

In some cases, there may be a continuous lumen through narrow regions 164 and wide regions 166. In some cases, narrow regions 164 may be sufficiently narrow to substantially seal the lumen between wide regions 166 to form compartments. Lumen barriers (as described above) placed in narrow regions 164 may be used to assist in sealing the compartments. Also, a core wire (as described below) contained in the lumen of the coil of minor windings may be used to assist in sealing the compartments. As explained above, where therapeutic agents are contained in the lumen of the coil of minor windings, such compartmentalization of the lumen may be useful in controlling the amount of therapeutic agent released.

In certain embodiments, the major windings of the elongate member have one or more flexible portions where the generally helical path taken by the elongate member is interrupted. These flexible portions may impart increased radial flexibility (e.g., increased compressibility or expandability) to the implant. This may be particularly useful where the implant is used in the superficial femoral artery, which can have lesions that are relatively long (e.g., extending for many centimeters) such that the implant must adapt to changes in the diameter of the artery as it traverses these relatively long lesions. At a flexible portion, the elongate member can deviate from the generally helical path in various suitable directions (e.g., taking a more transverse direction relative to the axis of the coil of major windings).

For example, at a flexible portion, the elongate member may take a path that forms bends, kinks, turns, spirals, fan-folds, or zig-zags. In some cases, the path taken by the elongate member at a flexible portion is limited to the plane defined by the major windings (e.g., a cylindrical or tubular plane). The number of flexible portions in the major windings will vary depending upon the particular application. In some cases, there are one or more flexible portions for every complete turn of the major windings. In some cases, there may be less than one flexible portion per complete turn (e.g., one flexible portion for every two complete turns of the major windings).

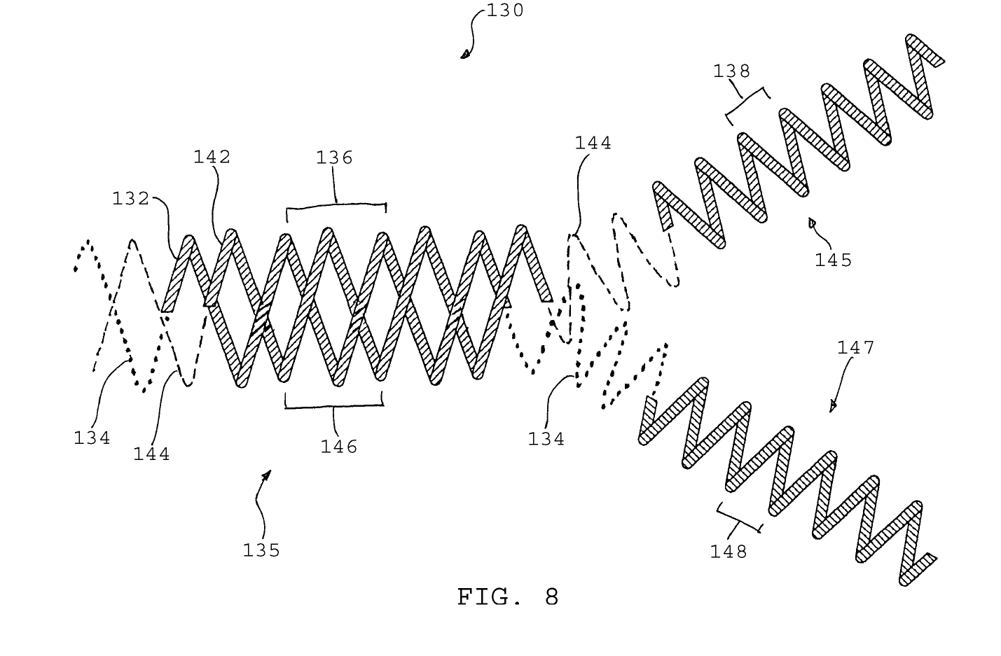

Referring to the embodiment shown in FIGS. 7A and 7B, a segment 144 of an implant comprises an elongate member 140 that, in its native configuration, follows a generally helical path to form major windings 148. Major windings 148 includes flexible portions 142 (one for each complete turn) where elongate member 140 deviates from the generally helical path and takes a zig-zag route before continuing on the generally helical path. As shown in the end view of FIG. 7B, the zig-zag route taken by elongate member 140 at flexible portions 142 is limited to the cylindrical plane of the major windings (which defines a lumen 146).

In certain embodiments, the implant comprises two or more elongate members, wherein each of the elongate members is wound into a coil of major windings. The two or more coils of major windings may define a singe lumen (i.e., the two or more coils of major windings share a common lumen), define different lumens, or a combination thereof. In some cases, the two or more coils of major windings may define a single lumen at a portion of the implant, and different lumens at another portion of the implant.

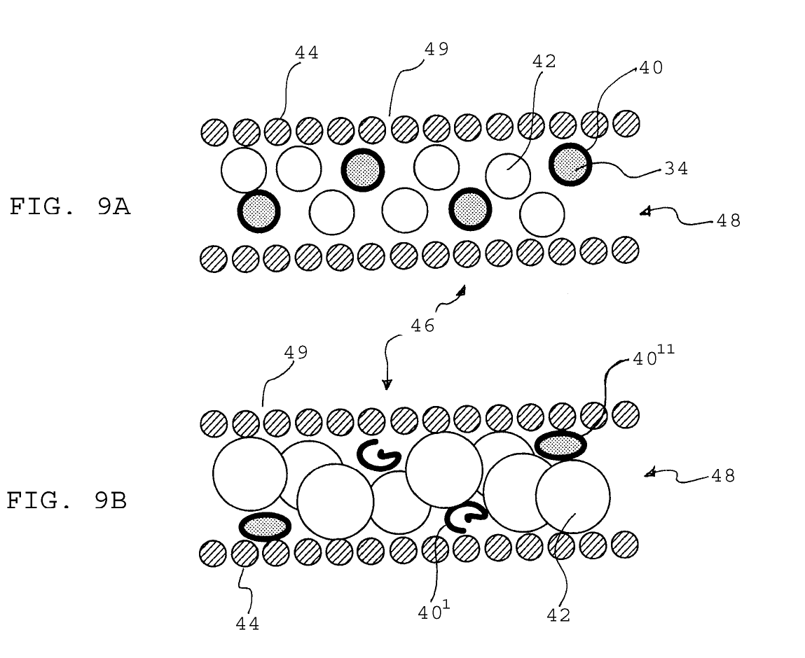

Referring to the embodiment shown in FIG. 8, a medical device comprises an implant 130 in the form of two elongate members, 132 and 142, with each of elongate members 132 and 142, in their native configurations, following a generally helical path to form major windings. Each of elongate members 132 and 142 comprises a wire coil that forms minor windings, the axis of which follows the generally helical path. For clarity, the path taken by the major windings of elongate member 132 is shown by dotted line 134, and the path taken by the major windings of elongate member 142 is shown by dashed line 144.

Implant 130 has a main body 135 at its proximal portion and two legs 145 and 147 extending distally from main body 135. Elongate member 132 forms major windings 136 at main body 135 of implant 130; and forms major windings 138 at leg 145 of implant 130. Elongate member 142 forms major windings 146 at main body 135 of implant 130; and forms major windings 148 at leg 147 of implant 130. Thus, at main body 135 of implant 130, major windings 136 of elongate member 132 and major windings 146 of elongate member 142 define a single common lumen.

Distal to main body 135, the axis of major windings 136 and the axis of major windings 146 begin to diverge and take different paths (with the path taken by major windings 136 shown by dotted line 134 and the path taken by major windings 146 shown by dashed line 144). Further distally, at legs 145 and 147 of implant 130, major windings 138 and major windings 148 define two different lumens.

Implant 130 may be useful in treating vascular disease that involves a branch point in the blood vessel (e.g., a bifurcation lesion). Main body 135 may be positioned in the blood vessel above the branch point, with one of legs 145 or 147 extending into the side branch of the blood vessel, while the other leg continues down the main trunk of the blood vessel.

Referring back to FIGS. 1A and 1B, in certain embodiments, lumen 15 of wire coil 12 contains capsules that hold a therapeutic agent. In some cases, the size of the capsules is larger than the width of the gaps between minor windings 14. In some cases, the capsules have a size in the range of 50 nm to 25 μm.

The capsules may be microspheres, liposomes, micelles, vesicles, or any of other various drug delivery particles that are known to be used for containing a therapeutic agent. For example, the capsules may be those described in commonly-assigned U.S. application Ser. No. 11/836,237 (Drug Delivery Device, Compositions and Method Relating Thereto) or U.S. Pat. No. 7,364,585 (Medical Devices Comprising Drug-Loaded Capsules for Localized Drug Delivery), both of which are incorporated by reference herein in their entirety. The capsules may have any of various shapes, including spherical shapes or irregular shapes. The capsules may be formed by the layer-by-layer self-assembly technique described in U.S. application Ser. No. 11/836,237 or U.S. Pat. No. 7,364,585, both of which are incorporated by reference herein in their entirety.

In some cases, the shell of the capsules may comprise any suitable polymer material that is biocompatible or otherwise known to be used in drug delivery particles. The polymer material may be biodegradable or bioerodible. Other suitable materials include ionic polymers, polyelectrolytes, biologic polymers, and lipids.

The capsules are designed to elute the therapeutic agent, and as such, may open, rupture, or become more permeable to the therapeutic agent when subject to mechanical stress (internal and/or external), resulting in the release of the therapeutic agent. Various properties of the capsules may be adjusted to provide this feature, including the capsule shell thickness, the number of shells, or the composition of the shells.

Furthermore, in some cases, the lumen may contain a swellable material that swells upon exposure to an aqueous environment (e.g., after implantation in the body). In such cases, swelling of the swellable material applies external pressure against the capsules, causing them to open, rupture, or become more permeable to the therapeutic agent such that the therapeutic agent is released from the capsules. The swellable material may be a hydrogel or other material that swells in volume upon absorption of water. Hydrogel materials include those disclosed in U.S. application Ser. No. 11/836,237 or U.S. Pat. No. 7,364,585 (both of which are incorporated by reference herein in their entirety), such as polyvinylpyrrolidine (PVP), polyethylene glycol (PEG), polyethylene oxide (PEO), and polyvinyl alcohols.

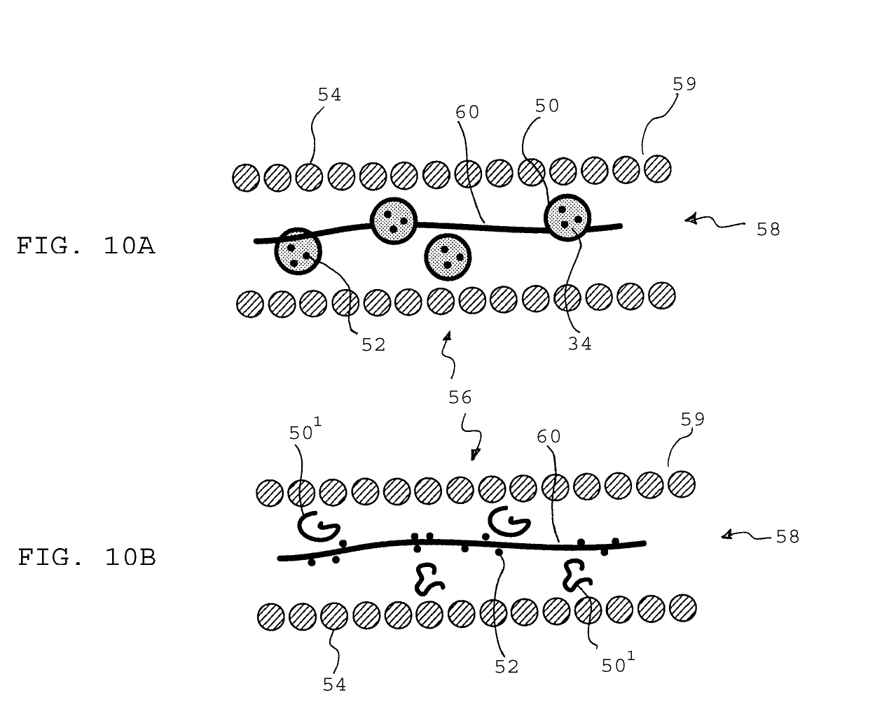

Referring to the embodiment shown in FIGS. 9A and 9B, a segment 46 of an implant comprises a wire coil 44 forming minor windings that define a lumen 48. Contained in lumen 48 are capsules 40 that contain a therapeutic agent 34. Also contained in lumen 48 are swellable particles 42 formed of a swellable material. FIG. 9A shows the medical device prior to implantation in a patient's body. Upon implantation, body fluid enters lumen 48 through the gaps 49 between the minor windings. As shown in FIG. 9B (after implantation), absorption of fluid by swellable particles 42 causes them to swell. Swollen particles 42 apply external pressure against capsules 40, causing them to become compressed (shown as compressed capsules 40″) or rupture (shown as ruptured capsules 40′) such that therapeutic agent is released.

Because capsules 40 are contained in lumen 48 of wire coil 44, in some embodiments, capsules 40 can be relatively large (in the range of 10-25 μm), which may otherwise be undesirable because of the risk of embolization. Also, because capsules 40 are contained in lumen 48 of wire coil 44, capsules 40 do not have direct contact with body tissue. This reduces any biocompatibility concerns that may otherwise be associated with the use of capsules 40. As such, in some embodiments, capsules 40 may comprise a polymer material that is not fully biocompatible (e.g., known to cause a significant inflammatory reaction or vascular thrombosis). This feature may be useful in extending the range of materials that can be used in capsules 40. This includes polymer materials that would be desirable to use, but otherwise avoided because of a lack of full biocompatibility. In such embodiments, capsules 40 may later degrade into non-toxic or low-toxicity substances that can be released out of lumen 48.

In an alternate embodiment, capsules 40 contain a swellable material in addition to containing therapeutic agent 34 (lumen 48 may or may not contain a swellable material). In this embodiment, the shell of capsules 40 are permeable, allowing fluid to penetrate into capsules 40. Internal pressure created by swelling of the swellable material causes the capsules to open, rupture, or other become more permeable to the therapeutic agent such that the therapeutic agent is released from capsules 40.

In another alternate embodiment, a corrodable element (e.g., a corrodable wire) is disposed in lumen 48 of wire coil 44, in which corrosion of the corrodable element raises or lowers the pH of the local environment within lumen 48. For example, the corrodable element may comprise magnesium, which generates hydroxide upon corrosion, thus raising the pH. Further, the swellable material may be a pH-sensitive polymer in which contraction or expansion of the polymer is triggered by a change in pH. Such pH-sensitive polymers include polyelectrolytes having ionizable weak acid or weak base groups, such as those described in M. R. Aguilar et al., Smart Polymers & Their Applications as Biomaterials, Topics in Tissue Engineering, ch. 6 (Biomaterials), vol. 3 (2007).

The corrodable element may have any of various dimensions and geometries, so long as it is contained within the lumen of the minor windings. For example, the corrodable element may be a wire that extends through the elongate member in the lumen of the minor windings.

The release rate of the therapeutic agent can be controlled by controlling the corrosion rate of the corrodable element. The rate of corrosion of the corrodable element will depend upon various factors, including its structure and composition. As such, the composition of the corrodable element can be selected to achieve a desired corrosion rate. For example, the corrosion rate of magnesium may be accelerated by mixing iron or copper with the magnesium. Also, the corrodable element may have a polymer coating to slow the corrosion rate.

In some embodiments, the shell or interior of the capsules may contain magnetically-sensitive particles. As used herein, “magnetically-sensitive particle” means a particle comprising a magnetically-sensitive material, such as paramagnetic or ferromagnetic substances (e.g., ferrous substances such as iron or steel). Release of the therapeutic agent contained in the capsules can be facilitated or modulated by the application of an electromagnetic field (including electric and magnetic fields) to the medical device. The source of the electromagnetic field may be located outside the patient's body or within the patient's body (e.g., intravascular), and may be provided by various apparatuses (e.g., an MRI apparatus). The electromagnetic field may be static or time-varying (e.g., oscillating or alternating) so as to generate an electromagnetic wave (e.g., RF or microwave).

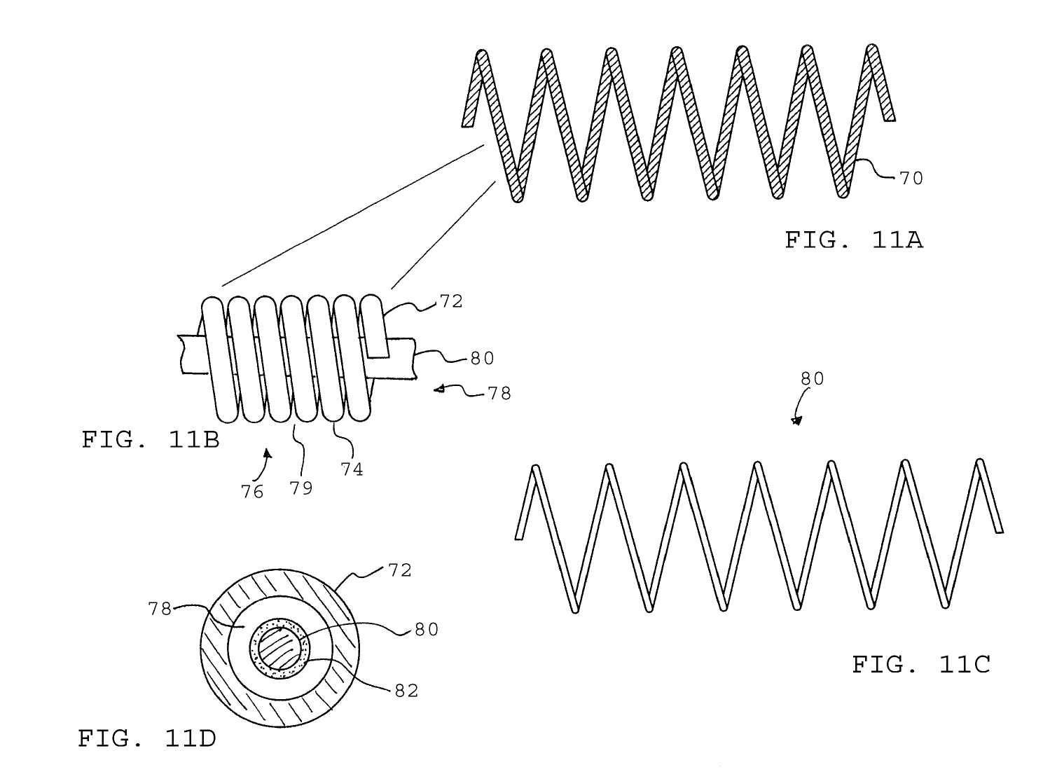

Referring to the embodiment shown in FIGS. 10A and 10B, a segment 56 of an implant comprises a wire coil 54 forming minor windings that define a lumen 58. Contained in lumen 58 are capsules 50 which contain a therapeutic agent 34. Also contained in capsules 50 are magnetically-sensitive magnetite particles 52. Furthermore, a magnetic wire 60 is contained in lumen 58 of wire coil 54.

FIG. 10A shows the medical device prior to implantation in a patient's body. After implantation, the medical device is subjected to an oscillating electromagnetic field applied from an external source. Under this oscillating electromagnetic field, magnetite particles 52 undergo vibrational motion and/or generate heat. As shown in FIG. 10B, this ruptures capsules 50 (shown as ruptured capsules 50′) or makes the capsules shells more permeable, such that therapeutic agent 34 is released from capsules 50. Therapeutic agent 34 is then released from lumen 58 through gaps 59 between the minor windings of wire coil 54. By magnetic attraction, magnetite particles 52 that are released from capsules 50 are drawn to and collected on magnetic wire 60. Magnetic wire 60 and/or magnetite particles 52 may later degrade into non-toxic or low-toxicity substances that are released out of lumen 58.

In certain embodiments, the lumen of the minor windings contains a core wire having a preset bias towards a generally helical configuration. By being contained in the lumen of the minor windings, the core wire biases the elongate member towards the generally helical configuration. The core wire may be formed of various materials capable of providing sufficient stiffness to bias the elongate member into a generally helical configuration. In some cases, the core wire may comprise a shape memory material, such as a shape memory metal (e.g., nitinol). In some cases, the core wire may comprise a polymer that is capable of providing sufficient stiffness to bias the elongate member into a generally helical configuration, including biodegradable polymers, such as biodegradable polyamide esters, biodegradable polycarbonates, or biodegradable polyurethane esters. Having the core wire comprised of a biodegradable polymer can be useful in allowing the implant to become more flexible or pliable after implantation when the core wire degrades. In some cases, the core wire may be coated with a therapeutic agent.

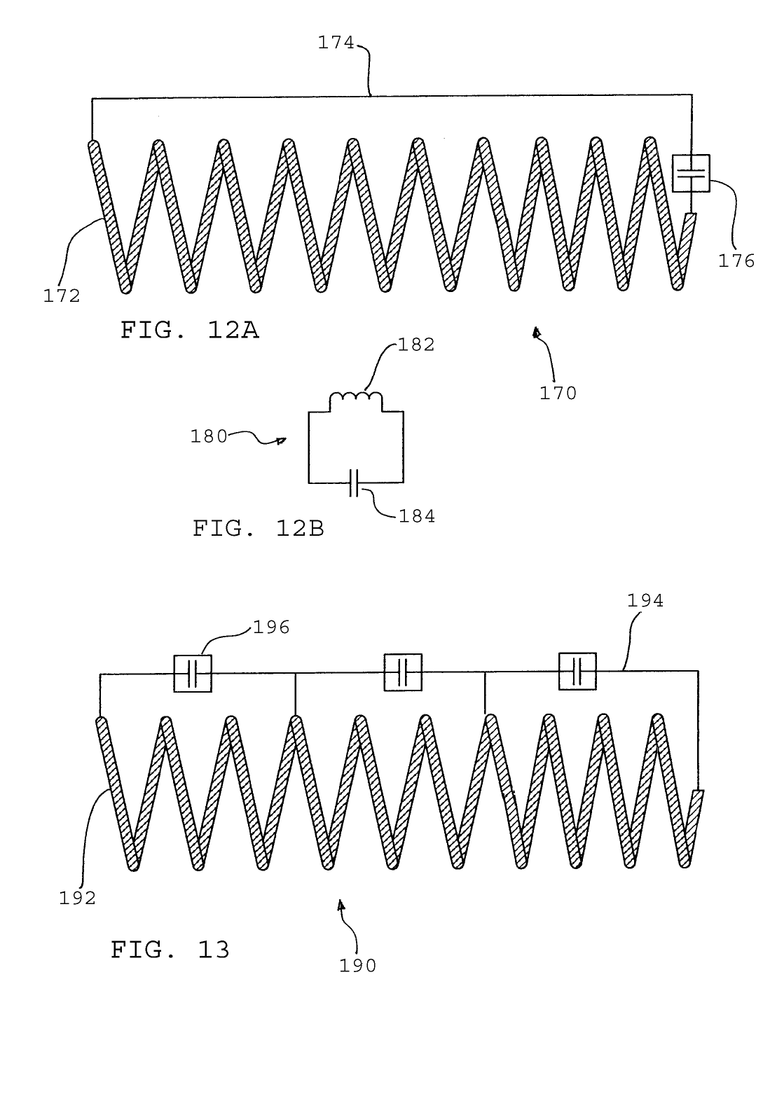

Referring to the embodiment shown in FIGS. 11A-11D, a medical device comprises an implant in the form of an elongate member 70 that, in its native configuration, follows a generally helical path to form major windings. FIG. 11B shows a detailed view of a segment 76 of elongate member 70. As seen in this view, elongate member 70 comprises a wire coil 72 forming minor windings 74 which define a lumen 78. A core wire 80 is contained in lumen 78 through the length of elongate member 70. As seen in FIG. 11C, core wire 80 has a preset bias towards a generally helical configuration. As such, core wire 80, contained in lumen 78 of elongate member 70, biases the shape of elongate member 70 towards a generally helical configuration. As seen in FIG. 11D, core wire 80 has a coating 82 containing a therapeutic agent. Upon implantation in a patient's body, the therapeutic agent contained in coating 82 is released through gaps 79 between minor windings 74.

After implantation, it may be desirable to image the implant using magnetic resonance imaging (MRI). However, in some cases, the electromagnetic properties of the implant (including possible magnetic field distortion or RF shielding caused by the composition and/or structure of the implant) may interfere with MR-imaging, resulting in poor quality images of the implant (e.g., image artifacts resulting from signal loss). The quality of the MR-generated images may be enhanced by adapting the implant to be capable of resonating at or close to the frequency of the RF energy applied by an MRI machine (e.g., at the Larmor frequency of the targeted atomic nuclei).

In certain embodiments, the medical device includes a resonance circuit with the coil of major windings serving as an inductor in the resonance circuit. This feature may be useful in allowing imaging of the implant by MRI. By having the resonance circuit tuned to the frequency of the RF energy applied by an MRI machine, improved visualization of the implant under MRI may be possible. Thus, adapting the coil of major windings to serve as an inductor in a resonance circuit can have the synergistic effect of allowing improved MR-imaging of the implant.

In some cases, the resonance circuit is tuned to resonate at a frequency in the range of 30-300 MHz. Having a resonant frequency in this range can be useful in allowing the implant to work with MRI machines that apply RF energy at Larmor frequencies suitable for hydrogen protons under magnetic field strengths conventionally used in MRI machines.

With the coil of major windings serving as an inductor, various suitable circuit configurations may be used to create a resonance circuit. In some cases, the resonance circuit includes one or more capacitance structures that are electrically coupled to the coil of major windings to form an inductance-capacitance (LC) circuit capable of resonating at a desired frequency. The resonant frequency of the LC circuit depends upon the inductance and the capacitance in the circuit. Thus, for a given inductance provided by the coil of major windings, the capacitance structure can be selected (e.g., according to its capacitance value) to provide a desired resonance frequency.

The capacitance structure may be any structure capable of providing capacitance to the resonance circuit and that is suitable for use with the implant. In some cases, the capacitance structure may be a discrete capacitor (e.g., a separate component). In some cases, the capacitance structure may include one or more portions of the elongate member (which may also be serving as an inductive element in the circuit) to form a structure providing capacitance. For example, one or more pairs of adjacent coils of the major windings of the elongate member may provide capacitance in the circuit (e.g., capacitance can be distributed in the coil of major windings). In another example, a terminal end of the elongate member may be included in the capacitance structure (e.g., to serve as an electrode plate). Capacitance structures and configurations for the resonance circuit can also be provided in the manner described in U.S. Application Publication No. 2008/0061788 by Weber (published 13 Mar. 2008), which is incorporated by reference herein in its entirety.

In some cases, the capacitance structure has an adjustable capacitance (e.g., a tunable capacitor). This feature can be useful in allowing adjustment of capacitance in the LC circuit to accommodate any changes in the inductance provided by the coil of major windings of the implant upon or after implantation (e.g., resulting from changes in the dimensions of the coil of major windings).

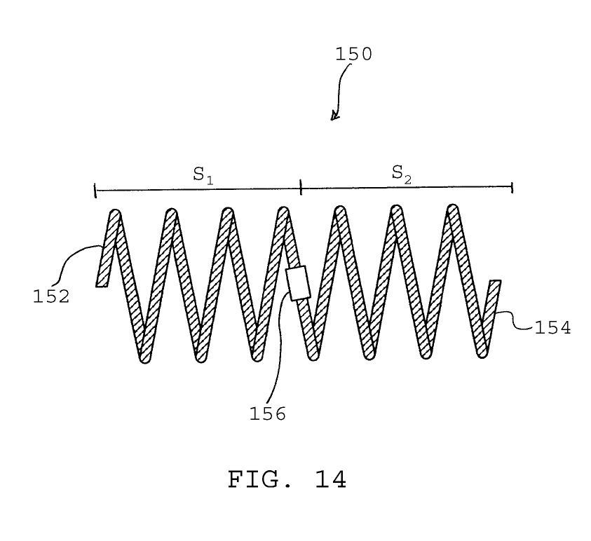

Referring to the embodiment shown in FIGS. 12A and 12B, a medical device comprises an implant 170 in the form of an elongate member 172 that, in its native configuration, follows a generally helical path to form major windings. Elongate member 172 comprises a wire coil forming minor windings. An electrically-conducting member 174 (e.g., a wire or shaft) is connected to the ends of implant 170 to form a closed circuit 180. The closed circuit includes a capacitor 176.

FIG. 12B shows a schematic diagram of closed circuit 180. In closed circuit 180, the major windings of elongate member 172 functions as an inductor 182. In closed circuit 180, capacitor 176 is represented by capacitor 184, which is selected according to its capacitance value such that closed circuit 180 is tuned to resonate at the frequency of the RF energy applied by an MRI machine.

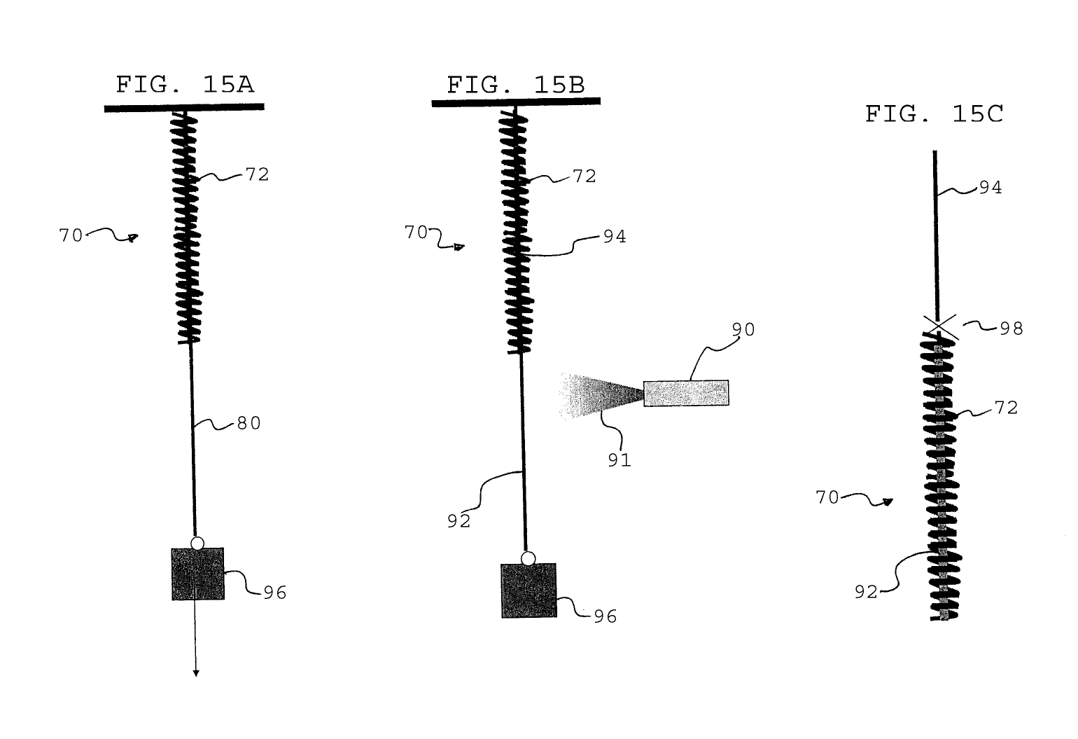

Other circuit configurations may also be used to form the resonance circuit. Referring to the embodiment shown in FIG. 13, a medical device comprises an implant 190 in the form of an elongate member 192 that, in its native configuration, follows a generally helical path to form major windings. Elongate member 192 comprises a wire coil forming minor windings. An electrically-conducting member 194 (e.g., a wire or shaft) is connected to the ends of implant 190 to form a closed circuit. Electrically-conductive member 194 is further connected to intermediate parts of implant 192, thus forming three parallel circuits. Each of the three parallel circuits has a capacitor 196. For each parallel circuit, capacitor 196 is selected to tune the individual parallel circuits to the frequency of the RF field generated by an MRI machine.

In certain embodiments, the implant is divided into segments that are electrically isolated from each other. When used with an MRI machine, the implant may act as a dipole antenna for the RF field emitted by the MRI machine. As such, this feature may be useful in reducing the effective antenna length of the implant to prevent the formation of a resonant standing RF wave in the implant when the implant is exposed to RF energy emitted by an MRI machine. A resonating standing RF wave in the implant can cause excessive heating or spark discharge at the ends of the implant. The problem of standing wave formation may be exacerbated for implants that are relatively long, such as those that are intended for use in the superficial femoral artery.

To avoid the formation of a standing RF wave, in some cases, one or more of the segments may have a length of less than ½ wavelength of the RF field experienced by the implant under MRI (taking into factor the wavelength compression resulting from the dielectric characteristics of body tissue through which the RF field must penetrate). For example, in some conventional 1.5 Tesla MRI machines, the implant can experience an RF field having a wavelength of about 26 cm. In such cases, dividing the implant into segments of less than 13 cm can avoid the formation of standing RF waves. The length of each of the segments may be the same or different from each other.

The segments may be electrically isolated from each other using any of various reactive circuit elements, including resistors (e.g., insulators), inductors, or capacitors. For example, insulating connectors made of a suitable non-conducting material (e.g., polymers or ceramics) may be used to separate the segments.

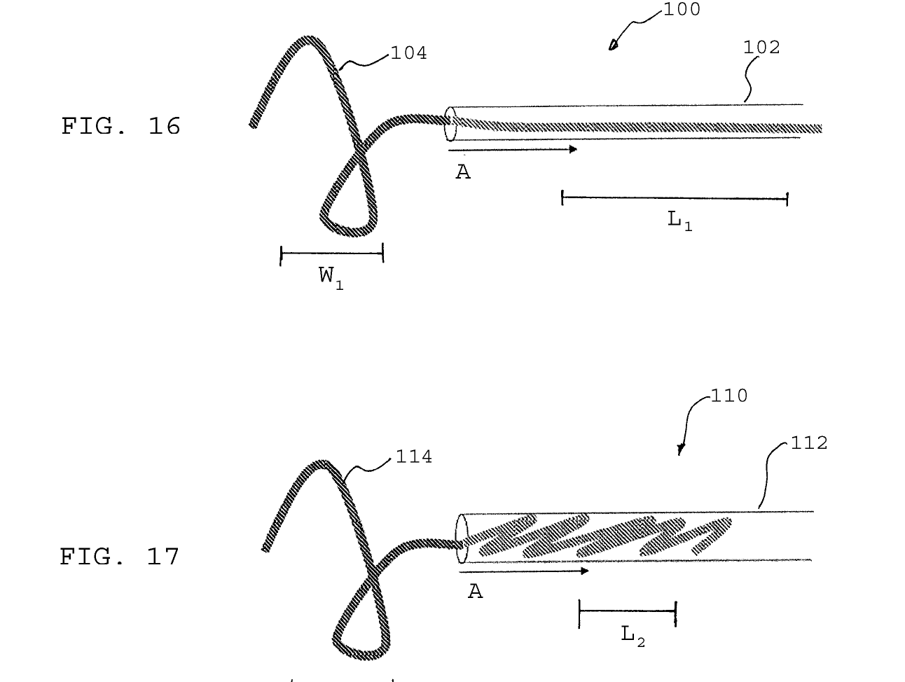

Referring to the embodiment shown in FIG. 14, a medical device comprises an implant 150. Implant 150 is formed of an elongate member 152 that is connected in series with elongate member 154. Both elongate members 152 and 154, in their native configurations, follow a generally helical path to form a coil of major windings. Elongate members 152 and 154 are connected to each other by an insulating connector 156 that divides implant 150 into two electrically isolated segments, whose lengths are represented by S₁ and S₂. Each of lengths S₁ and S₂ is less than 13 cm.

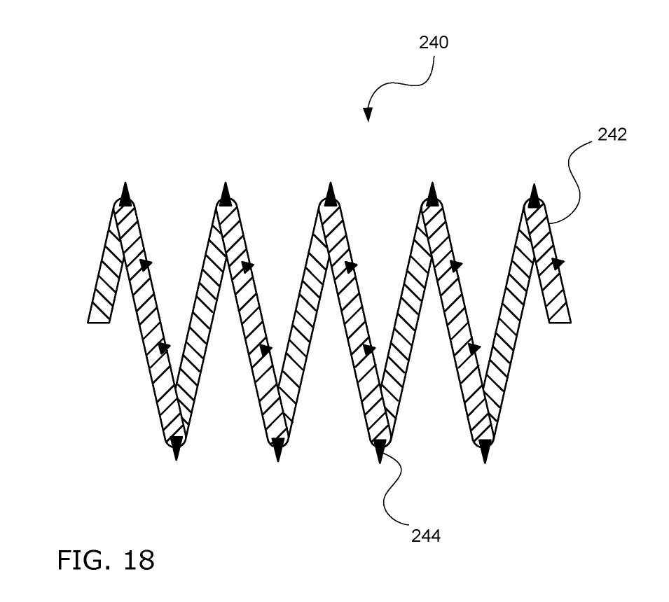

In another aspect, the present invention also provides a method of making a medical device. In one specific embodiment, referring to FIGS. 15A-15C, the method is for making the medical device shown in FIGS. 11A-11D above. The method involves providing elongate member 70 with core wire 80 disposed inside the lumen of wire coil 72. As seen in FIG. 15A, core wire 80 (which has a preset bias towards a helical configuration) is held in a straightened configuration (in this case, by attaching a weight 96 to an end of core wire 80). The length of core wire 80 at this stage is greater than the length of wire coil 72. When core wire 80 within wire coil 72 is straightened, wire coil 72 also assumes a straightened configuration. A portion 92 of core wire 80 is outside the lumen of wire coil 72, and another portion 94 is located inside the lumen of wire coil 72.

As seen in FIG. 15B, portion 92 of core wire 80 is coated with a therapeutic agent using any coating process known in the art (in this case, by using a spray nozzle 90 that creates a spray plume 91). Then, wire coil 72 is moved over to portion 92 (which is now coated with a therapeutic agent). Portion 94 of core wire 80 is then cut off at point 98. Then, core wire 80 is affixed to wire coil 72 (in this case, by laser welding the ends of core wire 80 to wire coil 72). When core wire 80 is released from its extended configuration, core wire 80 and wire coil 72 will return to the native configuration (i.e., generally helical).

In addition to the elongate member, medical devices of the present invention may further include components for delivering the elongate member to the target body site. For example, the medical device may be a system that includes a delivery catheter to deploy the elongate member into a blood vessel or other body lumen.

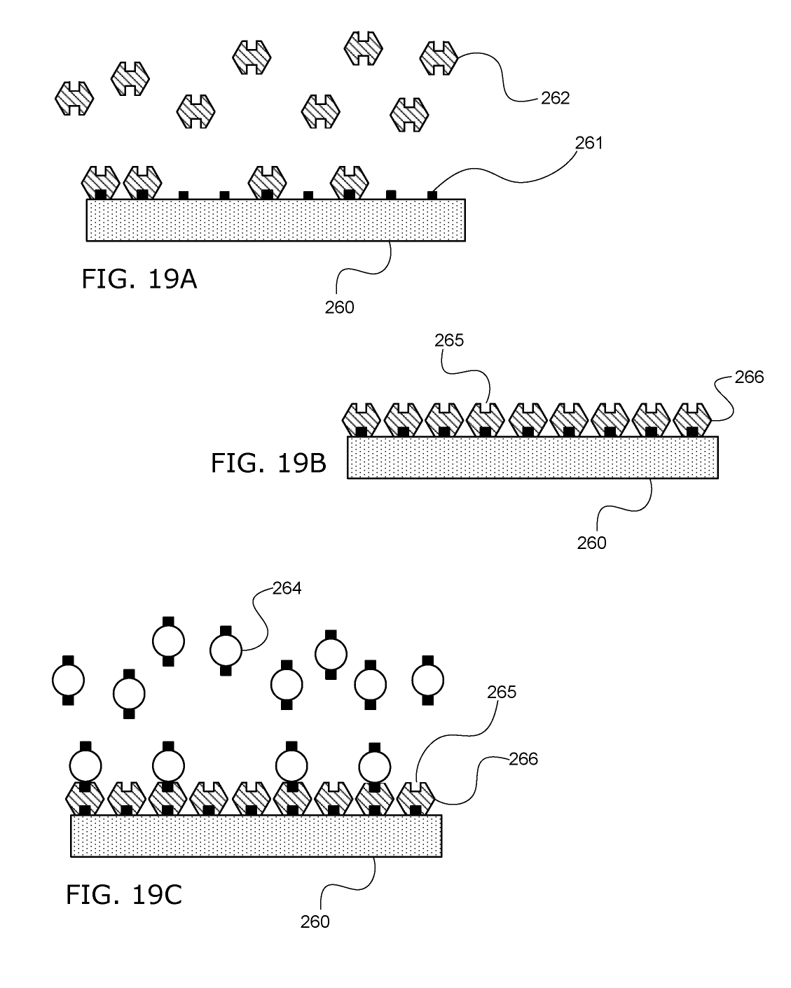

Referring to the embodiment shown in FIG. 16, a medical device 100 comprises a delivery catheter 102 having one or more lumens. An implant in the form of an elongate member 104 (which may be any of the elongate members described above) is contained within a lumen of delivery catheter 102. Within the lumen of delivery catheter 102, elongate member 104 is held in an extended configuration. Elongate member 104 may be released from the lumen of delivery catheter 102 by advancing elongate member 104 out of the catheter lumen and/or by retracting delivery catheter 102 in the direction of arrow A.

In FIG. 16, L₁ is the length of the portion of elongate member 104 that, when elongate member 104 is in its helical configuration, forms one major winding of width W₁. As seen in FIG. 16, length L₁ is greater than width W₁. As such, in some cases, the medical device further includes a mechanism for advancing elongate member 104 out of the catheter lumen as delivery catheter 102 is retracted such that retraction of delivery catheter 102 by a distance of W₁ will result in the release of a length L₁ of elongate member 104 to form a major winding of elongate member 104 (instead of needing to retract the delivery catheter a distance of L₁). The mechanism may comprise a pusher within the lumen of catheter 102. An actuator may be provided at the proximal end of catheter 102 to control delivery such that as the catheter is retracted by distance W₁, elongate member 104 is pushed out of catheter 102 by length L₁.

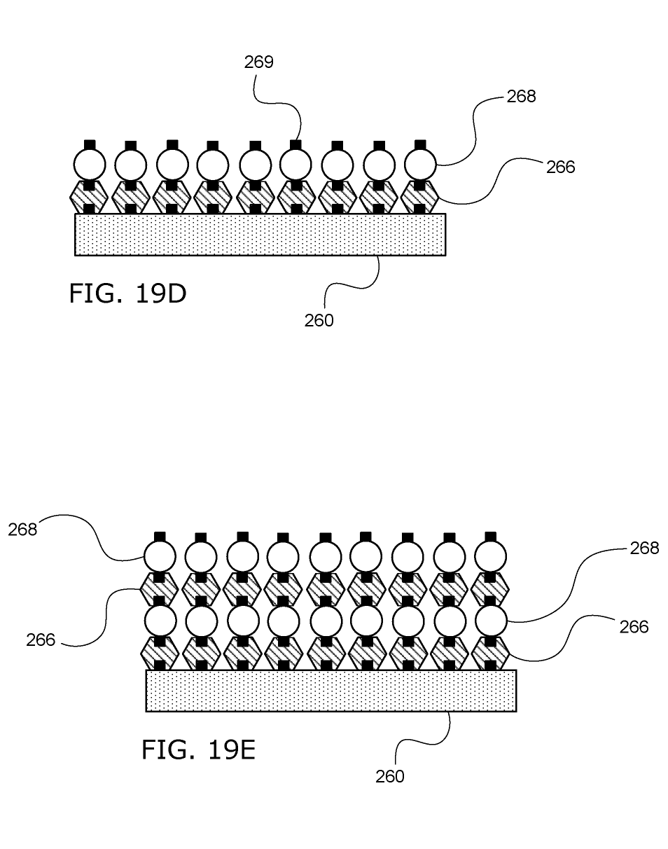

Referring to the embodiment shown in FIG. 17, a medical device 110 comprises a delivery catheter 112 having one or more lumens. An implant in the form of an elongate member 114 (which may be any of the elongate members described above) is contained within the lumen of delivery catheter 112. Within the lumen of delivery catheter 112, elongate member 114 is held in a compact, folded configuration. In this folded configuration, the width L₂ of each fold is substantially the same as the width W₂ of a major winding of elongate member 114 in the helical configuration. As such, retraction of delivery catheter 112 by a distance of W₂ will result in the release of a major winding of elongate member 114 (i.e., such that there is a one-to-one ratio in the distance of catheter retraction to the length of elongate member 114 that is released from the catheter).

In some cases, in the medical device of FIG. 17, instead of being loaded into delivery catheter 112 in a folded configuration, elongate member 114 may be loaded into delivery catheter 112 in a compact coiled configuration, which has windings that are more compact than the major windings (when elongate member 114 is in its native configuration). When elongate member 114 is released from delivery catheter 112, elongate member 114 unwinds from its compact coiled configuration into the coil of major windings.

In some cases, the compact coiled configuration may include turns in opposite directions. This feature can be useful in reducing the amount of torsional force being applied to the surrounding tissue as the compact coiled configuration unwinds. For example, in the compact coiled configuration, a series of clockwise turns may be followed by a series of counter-clockwise turns (or vice versa). Further reduction in torsional force may be achieved by having the number of clockwise turns be the same or close to the number of counter-clockwise turns. For example, where each of the major windings constitutes 7 turns in the compact coiled configuration, the compact coiled configuration may have windings with 4 clockwise turns followed by 3 counter-clockwise turns (and then followed by 3 clockwise turns and 4 counter-clockwise turns, and so on).

Referring back to FIGS. 1A and 1B, in certain embodiments, lumen 15 of wire coil 12 contains particles that carry a therapeutic agent. The particles may be designed to prevent their escape out of lumen 15. For example, the particles may have a size larger than the gaps between windings 14. For example, the particles may have an average size of 10 μm or greater, and in some cases, have an average size in the range of 10-100 μm. Other particle sizes are also possible, depending upon the particular application.

In some cases, the particles are formed of an inorganic material. The inorganic material may be a ceramic-type material (e.g., silicon oxide or a metal oxide, such as aluminum oxide) or a metal, such as iron, magnesium, zinc, aluminum, gold, silver, titanium, manganese, iridium, or alloys of such metals. In some cases, the metals may be selected from those that are biodegradable or bioresorbable, such as iron, magnesium, zinc, or alloys of such metals. The particles may be solid or porous (e.g., porous silicon oxide particles). Solid particles may be coated with the therapeutic agent, whereas porous particles may be loaded with the therapeutic agent in the pores.

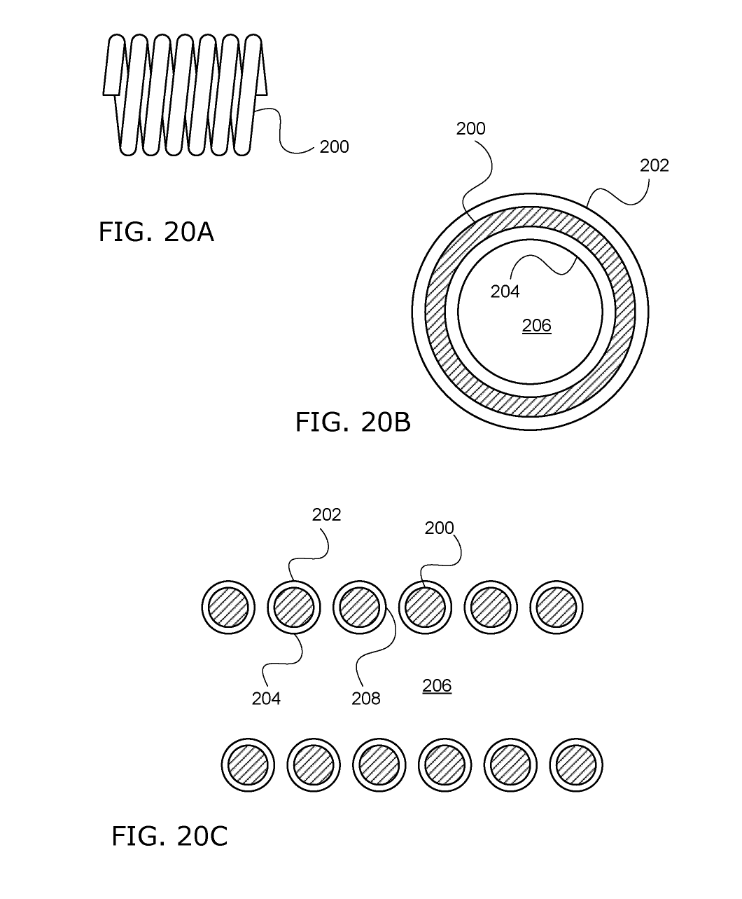

In certain embodiments, an implant of the present invention has anchors as described in U.S. Patent Application Publication No. 2009/0043276 (by Jan Weber, for application Ser. No. 11/836,237) titled “Drug Delivery Device, Compositions And Methods Relating Thereto,” which is incorporated by reference herein. For example, referring to the embodiment shown in FIG. 18, a medical device comprises an implant 240 in the form of an elongate member 242 that follows a generally helical path. Elongate member 242 comprises a wire coil forming minor windings. Elongate member 242 has anchors 244 for securing implant 240 to the body tissue. Anchors 244 may be configured as nails, hooks, tacks, pins, and the like. Anchors 244 may be biostable, bioerodable, or biodegradable. In some cases, anchors 244 are formed of a bioerodable or biodegradable metal, such as magnesium or iron. Anchors 244 may be attached to elongate member 242 by a biocompatible adhesive.

In certain embodiments, the implants of the present invention have a coating that is deposited by a self-limiting deposition process. In a self-limiting deposition process, the growth of the coating stops after a certain point (e.g., because of thermodynamic conditions or the bonding nature of the molecules involved), even though sufficient quantities of deposition materials are still available. For example, the coating may grow in a layer-by-layer process where the growth of each monolayer is completed before the next monolayer is deposited.

The present invention may use any of various types of self-limiting deposition processes suitable for coating the implant. Examples of self-limiting deposition processes include atomic layer deposition (also known as atomic layer epitaxy), pulsed plasma-enhanced chemical vapor deposition (see Seman et al., Applied Physics Letters 90:131504 (2007)), molecular layer deposition, and irradiation-induced vapor deposition.

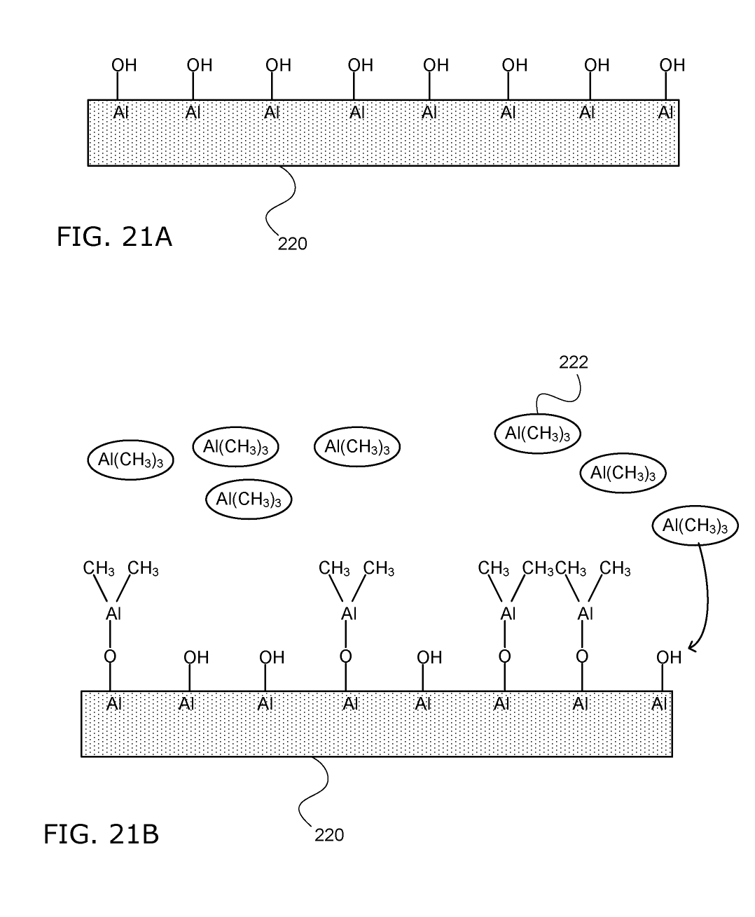

Atomic layer deposition is a gas-phase deposition process in which a coating is grown onto a substrate by self-limiting surface reactions. Atomic layer deposition is commonly performed using a binary reaction sequence, with the binary reaction being separated into two half-reactions. FIGS. 19A-E schematically illustrate an example of how a coating can be formed by atomic layer deposition using two sequential half-reactions. Referring to FIG. 19A, a substrate 260 with a surface having reactive sites 261 is placed inside a reaction chamber. In the first half-reaction, a first precursor species 262 in vapor phase is fed into the reaction chamber. First precursor species 262 is chemisorbed onto the surface of substrate 260 by reacting with reactive sites 261. As shown in FIG. 19B, the chemisorption of precursor species 262 proceeds until saturation of the surface, at which point, the reaction self-terminates, resulting in a monolayer 266. Once this half-reaction is completed, additional reactant exposure produces no additional growth of monolayer 266. The reaction chamber is then purged of first precursor species 262. Monolayer 266 has reactive sites 265 for reacting with the next precursor material.

As shown in FIG. 19C, for the second half-reaction, a second precursor species 264 in vapor phase is fed into the reaction chamber. Second precursor species 264 reacts with reactive sites 265 on the surface of monolayer 266. As shown in FIG. 19D, the chemisorption of second precursor species 264 proceeds until saturation of monolayer 266, at which point, the reaction self-terminates, resulting in another monolayer 268. The reaction chamber is then purged of second precursor species 264. The surface of monolayer 268 has reactive sites 269 capable of reacting with first precursor species 262, allowing additional reaction cycles until the desired coating thickness is achieved. For example, FIG. 19E shows substrate 260 having a series of monolayers 266 and 268 formed by several reaction cycles.

By using a self-limiting deposition process to coat the implant, the coating can have more uniformity in thickness across different regions of the implant and/or a higher degree of conformality. Other coating processes (e.g., line-of-sight deposition processes, such as spray coating) may only have limited ability to coat the more spatially challenging surfaces of the implant, such as the luminal surface (facing internally) of the coil of minor windings or the interspace between the minor windings. This could result in the unequal build-up of coating. For example, the coating on the external surface of the coil of minor windings may end up being thicker than the coating on the luminal surface.

By using a self-limiting deposition process, a more uniform coating thickness on the implant may be possible. Also, it has been demonstrated that very high aspect ratio structures (such as deep and narrow trenches or nanoparticles) can be coated uniformly by atomic layer deposition. As such, using a self-limiting deposition process may allow for the coating of even less accessible parts of the implant, such as surfaces in the spaces between the minor windings of the coil. This could result in a more conformal coating for the implant.

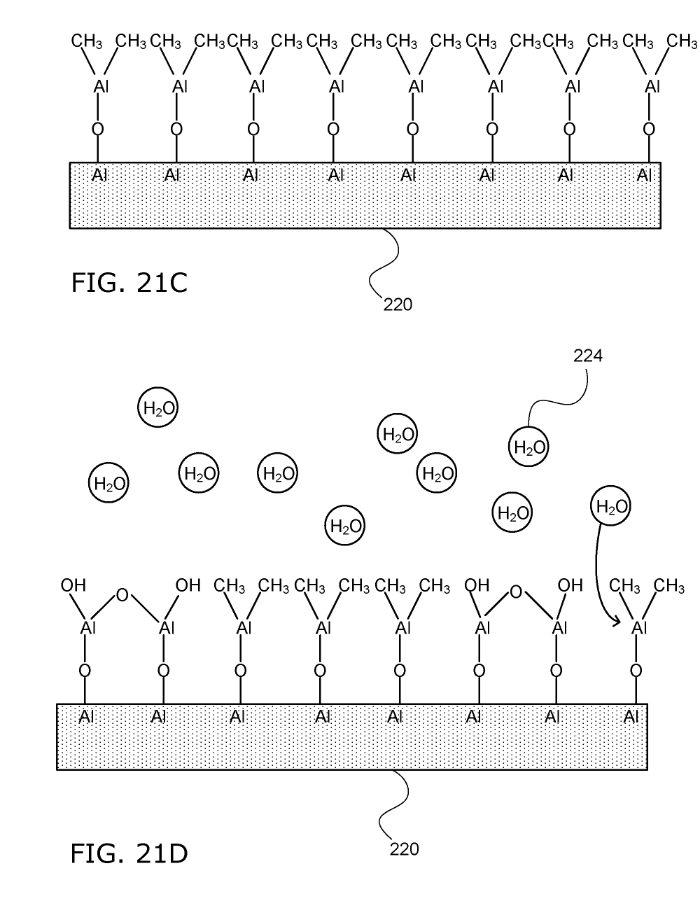

For example, FIGS. 20A-C show an implant having a coating deposited using atomic layer deposition. FIG. 20A shows a side view of a portion of the implant, which comprises a wire coil 200 forming minor windings. As shown in the end view of FIG. 20B, the implant has a lumen 206 defined by the minor windings of wire coil 200. As also seen in this view, the implant has an inner coating 204 on the luminal side of the coil of minor windings and an outer coating 202 on the external side of the coil of minor windings. Atomic layer deposition of the coating can provide a more uniform coating thickness on the implant. As such, the thickness of inner coating 204 as compared to the thickness of the outer coating 202 can differ, for example, by less than 20% of the thickness of the outer coating (e.g., the inner coating may be thinner), or in some cases, can differ by less than 10%, or in some cases, can be substantially the same. FIG. 20C shows a longitudinal cross-section view of the portion of the implant shown in FIG. 20A. This view shows outer coating 202 and inner coating 204 from a different perspective. This view also shows an interspace coating 208 on the wire coil 200 between the minor windings. Outer coating 202, inner coating 204, and interspace coating 208 together form a conformal coating. The thickness of interspace coating 208 as compared to the thickness of the inner coating or the outer coating can differ, for example, by less than 20% of the thickness of the inner coating or outer coating (e.g., the interspace coating may be thinner), or in some cases, can differ by less than 10%, or in some cases, can be substantially the same.

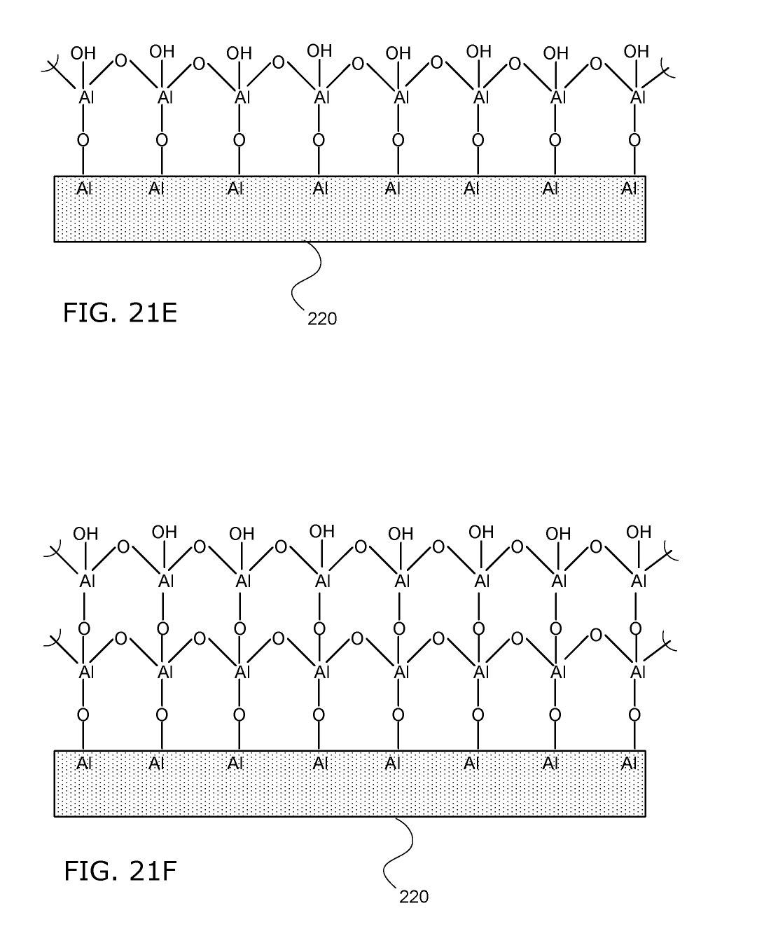

In some cases, the self-limiting deposition process is used to deposit an inorganic coating on the implant. For example, FIGS. 21A-F demonstrate how an aluminum oxide coating may be formed on the implant by atomic layer deposition. The process involves the following two sequential half-reactions:

:Al—OH+Al(CH₃)_{3(g)}→:Al—O—Al(CH₃)₂+CH₄ (A)

:Al—O—Al(CH₃)₂+2H₂O→:Al—O—Al(OH)₂+2CH₄ (B)

with Al—OH and :Al—O—Al(CH₃)₂ being the surface species. These two half-reactions give the overall reaction :Al—OH+Al(CH₃)₃+2H₂O→:Al—O—Al(OH)₂+3CH₄.

FIG. 21A shows a portion 220 of an aluminum implant providing an aluminum surface having native hydroxyl groups. These native hydroxyl groups may be provided by pretreatment of the aluminum surface with water vapor. Referring to FIG. 21B, the implant is placed inside a reaction chamber and Al(CH₃)₃ (trimethylaluminum) gas is introduced into the reaction chamber. The Al(CH₃)₃ molecules react with the native hydroxyl groups on the aluminum surface to form a methyl-terminated aluminum species. Referring to FIG. 21C, after all the native hydroxyl groups are reacted with Al(CH₃)₃, the reaction self-terminates, resulting in a monolayer of methyl-terminated aluminum. The reaction chamber is then purged of the excess Al(CH₃)₃ gas.

Next, water vapor is introduced into the reaction chamber. As shown in FIG. 21D, the water molecules 224 react with the dangling methyl groups on the new monolayer surface to form Al—O bridges and surface hydroxyl groups. Referring to FIG. 21E, after all the methyl-terminated aluminum species are reacted with the water molecules 224, the reaction self-terminates, resulting in a monolayer of aluminum hydroxide species. This monolayer of aluminum hydroxide species has hydroxyl groups that are ready for the next cycle of exposure to trimethylaluminum. Referring to FIG. 21F, these reactions are repeated in a cyclic manner to form a coating of the desired thickness. This type of atomic layer deposition is available at Beneq (Vantaa, Finland).

Atomic layer deposition can be used to deposit numerous types of materials, including both inorganic and organic materials. For example, besides Al₂O₃, atomic layer deposition coating schemes have been designed for silica (SiO₂), silicon nitride (Si₃N₄), titanium oxide (TiO₂), boron nitride (BN), zinc oxide (ZnO), tungsten (W), and others. Also, it is known that an iridium oxide coating can be deposited by atomic layer deposition using an alternating supply of (ethylcyclopentadienyl)(1,5-cyclooctadiene)iridium and oxygen gas at temperatures between 230 to 290° C. Other inorganic materials that could be deposited using atomic layer deposition include B₂O₃, Co₂O₃, Cr₂O₃, CuO, Fe₂O₃, Ga₂O₃, HfO₂, In₂O₃, MgO, Nb₂O₅, NiO, Pd, Pt, SnO₂, Ta₂O₅, TaNx, TaN, AlN, TiCrOₓ, TiN, VO₂, WO₃, ZnO, (Ta/Al)N, (Ti/Al)N, (Al/Zn)O, ZnS, ZnSe, ZrO, Sc₂O₃, Y₂O₃, Ca₁₀(PO₄)(OH)₂ (hydroxylapatite), and rare earth oxides. Atomic layer deposition has also been used with organic materials, including 3-(aminopropyl)trimethoxysiloxane and polyimides, such as 1,2,3,5-benzenetetracarboxylic anhydride-4,4-oxydianiline (PMDA-ODA) and 1,2,3,5-benzenetetracarboxylic anhydride-1,6-diaminohexane (PMDA-DAH).

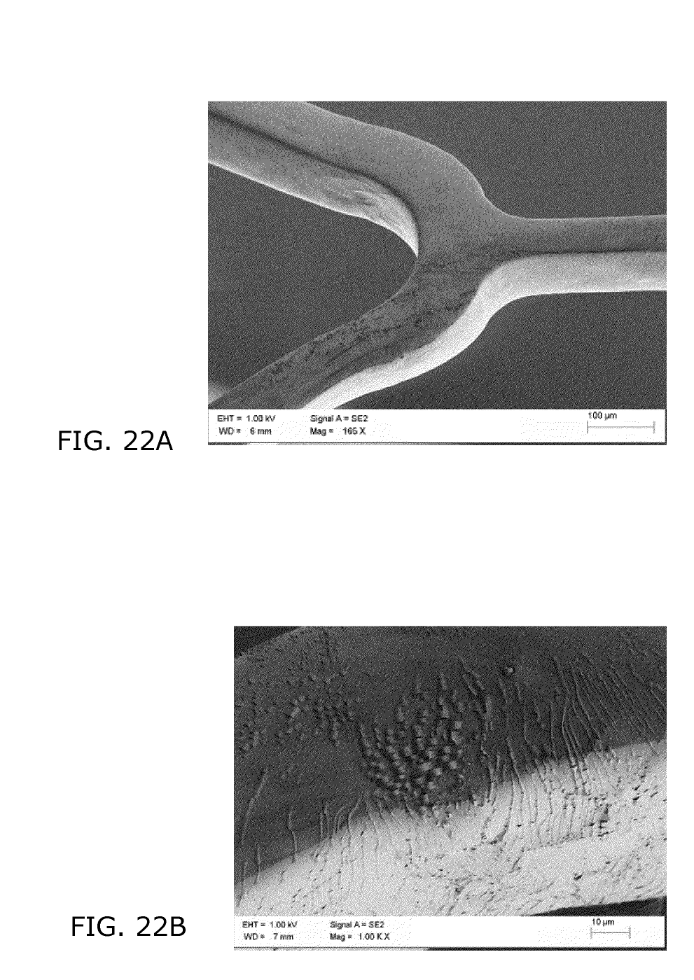

The coating formed by the self-limiting deposition process may have various thicknesses, depending upon the particular application. For FIGS. 22A and 22B, coronary artery stents were coated with titanium oxide by atomic layer deposition at 80° C. to a thickness of either 5 nm or 30 nm. FIG. 22A shows a microscopic image of the stent having the 5 nm thick titanium oxide coating, with the image taken after expansion of the stent. As seen here, there was no visible cracking or delamination of the titanium oxide coating. FIG. 22B shows a microscopic image of the stent having the 30 nm thick titanium oxide coating, with the image taken after expansion of the stent. As seen here, there was some cracking and delamination of the coating at high strain points after expansion of the stent. Based on these results, in some embodiments, such as a stent as in FIGS. 22A and B, the thickness of the inorganic coating is less than 30 nm, and in some cases, less than 20 nm.

The coating formed by the self-limiting deposition process may be inorganic or organic. In certain embodiments, the coating is inorganic, and in some cases, the inorganic coating may comprise a material that is capable of undergoing a photocatalytic effect such that the coating becomes superhydrophilic. For example, titanium oxide coatings can be made superhydrophilic and/or hydrophobic using the technique described in U.S. Patent Application Publication No. 2008/0004691 titled “Medical Devices With Selective Coating” (by Weber et al., for application Ser. No. 11/763,770), which is incorporated by reference herein. For example, after a titanium oxide coating is applied onto an implant, the implant can be placed in a dark environment to cause the titanium oxide coating to become hydrophobic, followed by exposure of the coating (or selected portions of the coating) to UV light to cause the coating (or selected portions) to become superhydrophilic (i.e., such that a water droplet on the coating would have a contact angle of less than 5°). Superhydrophilic coatings can be useful for carrying therapeutic agents, providing a more biocompatible surface for the implant, and/or promoting adherence of endothelial cells to the implant.

By selectively making some portions of the coating more hydrophilic or hydrophobic relative to other portions, it may be possible to selectively apply other materials, such as drugs or other coating materials, onto the implant based on the hydrophilicity or hydrophobicity of these other materials. For example, referring back to FIGS. 20A-C, the inner coating 204 can be made superhydrophilic by UV light exposure through a fiber optic line inserted within the lumen 206 of wire coil 200, or the outer coating 202 can be made superhydrophilic by exposing the exterior of wire coil 200 to UV light. A hydrogel coating containing a therapeutic agent can then be applied onto the superhydrophilic portions of the coating.

Medical devices of the present invention may have any of various applications. For example, the medical devices may be used as implants in blood vessels, including the superficial femoral artery. The medical devices could also be used in the coronary arteries, other peripheral arteries, or other body lumens.

The therapeutic agent used in the present invention may be any pharmaceutically acceptable agent (such as a drug), a biomolecule, a small molecule, or cells. Exemplary drugs include anti-proliferative agents such as paclitaxel, sirolimus (rapamycin), tacrolimus, everolimus, biolimus, and zotarolimus. Exemplary biomolecules include peptides, polypeptides and proteins; antibodies; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Exemplary small molecules include hormones, nucleotides, amino acids, sugars, and lipids and compounds have a molecular weight of less than 100 kD. Exemplary cells include stem cells, progenitor cells, endothelial cells, adult cardiomyocytes, and smooth muscle cells.

The foregoing description and examples have been set forth merely to illustrate the invention and are not intended to be limiting. Each of the disclosed aspects and embodiments of the present invention may be considered individually or in combination with other aspects, embodiments, and variations of the invention. In addition, unless otherwise specified, the steps of the methods of the present invention are not confined to any particular order of performance. Modifications of the disclosed embodiments incorporating the spirit and substance of the invention may occur to persons skilled in the art, and such modifications are within the scope of the present invention.

What is claimed is:

1. A medical device comprising:
an implant comprising a strand wound into a coil of minor windings about an axis, wherein the minor windings define a lumen, and wherein the coil of minor windings is wound into a coil of major windings such that the axis of the minor windings follows a generally helical path;
an inner coating disposed over the luminal surface of the coil of minor windings; and
an outer coating disposed over the external surface of the coil of minor windings;
wherein the thickness of the inner coating and the thickness of the outer coating are substantially the same or differ by less than 20% of the thickness of the outer coating.

2. The medical device of claim 1, wherein the inner coating and the outer coating both comprise an inorganic material.

3. The medical device of claim 2, wherein the inorganic material is a metal oxide.

4. The medical device of claim 1, further comprising an interspace coating on the strand between the minor windings.

5. The medical device of claim 4, wherein the implant has a conformal coating that comprises the inner coating, the outer coating, and the interspace coating.

6. The medical device of claim 1, wherein the outer coating has a thickness of less than 30 nm.

7. A method of making a medical device, comprising:
providing an implant comprising a strand wound into a coil of minor windings about an axis, wherein the minor windings define a lumen, and wherein the coil of minor windings is wound into a coil of major windings such that the axis of the minor windings follows a generally helical path; and
depositing a coating over the implant using a self-limiting deposition process.

8. The method of claim 7, wherein the self-limiting deposition process is atomic layer deposition.

9. The method of claim 7, wherein the coating has a thickness of less than 30 nm.

10. The method of claim 7, wherein the coating comprises an inorganic material.

11. The method of claim 10, wherein the coating comprises titanium oxide, and wherein the method further comprises exposing at least a portion of the coating to UV light.

12. A medical device comprising:
an implant comprising a strand wound into a coil of minor windings about an axis, wherein the minor windings define a lumen, and wherein the coil of minor windings is wound into a coil of major windings such that the axis of the minor windings follows a generally helical path; and
a core wire disposed in the lumen of the coil of minor windings, wherein the core wire biases the coil of minor windings such that the axis of the minor windings follow the generally helical path;
wherein the improvement comprises a coating disposed on the core wire, wherein the coating comprises a therapeutic agent.

13. The medical device of claim 12, wherein the diameter of the lumen of the coil of minor windings is 200 μm or less.

14. The medical device of claim 13, wherein the diameter of the core wire is 100 μm or less.

15. The medical device of claim 12, wherein the core wire comprises nitinol.

16. A method of treating a superficial femoral artery comprising:
providing a medical device, wherein the medical device comprises an implant comprising a strand wound into a coil of minor windings about an axis, wherein the minor windings define a lumen that contains a therapeutic agent, and wherein the coil of minor windings is wound into a coil of major windings such that the axis of the minor windings follows a generally helical path; and
implanting the implant in the superficial femoral artery.

17. The method of claim 16, wherein the medical device further comprises a delivery catheter having a catheter lumen, wherein the implant is contained in the catheter lumen, and wherein the step of implanting comprises retracting the catheter to release the implant from the catheter lumen.

18. The method of claim 17, wherein retracting the catheter a distance that is substantially equal to the width of each of the major windings results in the release of a major winding of the implant from the catheter lumen.

19. The method of claim 16, wherein the diameter of the lumen of the coil of minor windings is 200 μm or less.

20. The method of claim 16, wherein the length of the coil of major windings is 2 cm-40 cm.

21. The method of claim 16, wherein the diameter of the coil of major windings is 3 mm-10 mm.
